(19) United States
(12) Patent Application Publication
(10) Pub. No.: US 2025/0387622 A1
(43) Pub. Date: Dec. 25, 2025

(54) WEARABLE DEVICE FOR REGAINING SIMULTANEOUS EYELID CLOSURE FOR VICTIMS OF PARTIAL FACIAL PARALYSIS VIA DIRECT STIMULATION

(71) Applicant: Georgia Tech Research Corporation, Atlanta, GA (US)

(72) Inventors: Ilija Uzelac, Atlanta, GA (US); Chris Crowley, Atlanta, GA (US); Harry Michael Baddour, Atlanta, GA (US)

(21) Appl. No.: 18/997,930

(22) PCT Filed: Aug. 4, 2023
(86) PCT No.: PCT/US2023/071715

§ 371 Date: Jan. 23, 2025

Related U.S. Application Data
(60) Provisional application No. 63/395,443, filed on Aug. 5, 2022.

(51) Int. Cl. A61N 1/36; A61N 1/04

(52) U.S. Cl. CPC A61N 1/36003; A61N 1/0452; A61N 1/0476; A61N 1/36025; A61N 1/36031; A61N 1/36034

(57) ABSTRACT

An exemplary embodiment of the present disclosure provides an eyelid closure system comprising a first optical sensor, an electrical stimulator, and a controller. The controller can be configured to: receive a sensing signal from the first optical sensor, the sensing signal indicative of a kinematic of a first eyelid of a user; and cause the electrical stimulator to emit a first electrical stimulus in response to the sensing signal, the first electrical stimulus configured to cause a second eyelid of the user to close.

### CROSS-REFERENCE TO RELATED APPLICATIONS

This application claims the benefit of U.S. Provisional Application Ser. No. 63/395,443 filed on 5 Aug. 2022, which is incorporated herein by reference in its entirety as if fully set forth below.

### FIELD OF THE DISCLOSURE

The various embodiments of the present disclosure relate generally to medical systems, and more particular to systems for stimulating eyelid closure.

### BACKGROUND

Blinking and eyelid closure are dependent on normal function of the facial nerve and its associated musculature. Disorders causing facial nerve dysfunction lead to weakness or paralysis of the facial muscle (orbicularis oculi) responsible for eyelid closure, which is clinically known as lagophthalmos. The inability to blink and achieve complete eyelid closure can lead to chronic eye irritation and pain. This is secondary to the disruption of the normal tearing mechanism as well as chronic exposure of the eye to debris and drying. In some cases, this can lead to corneal damage, vision loss and even blindness. There are numerous causes of acute facial paralysis; however, the most common cause is Bell's palsy, which is considered to be virally-mediated. Other causes of paralysis include acoustic tumor resections, trauma, surgically-related facial nerve injuries, Lyme disease and other related infectious causes. Typical manifestations include unilateral, rapid-onset facial weakness or paralysis causing significant facial asymmetry. Blink asymmetry/dysfunction and lagophthalmos are typical sequelae causing significant psychosocial and functional impairments. Bell's palsy has an incidence of ranging from 25 to 53.3 per 100,000 persons depending on the population. The incidence is highest between the ages of 15 to 45 years. Seventy to ninety percent of Bell's palsy patients recover completely with the remainder having residual symptoms, including poor blink restoration in many cases. Such sequelae of incomplete recovery can also be seen in the other causes mentioned above. Therefore, there exists a large population of patients that are affected by the inability to blink on both an acute and chronic basis secondary to facial paralysis resulting from a variety of etiologies.

Neuromuscular electrical stimulation (NMES) has been widely adopted in neuromuscular rehabilitation programs for prevention of muscle atrophy and promotion of muscular hypertrophy, composition and blood flow. This form of stimulation utilizes electrical energy to stimulate either motor nerves or muscle. Currently, there are no described commercially available, non-invasive or invasive, neuromuscular devices that have demonstrated the ability to restore spontaneous blinking and eyelid closure in the setting of facial paralysis. A need therefore exists for such a device that can assist with blink restoration in this population.

### BRIEF SUMMARY

An exemplary embodiment of the present disclosure provides an eyelid closure system comprising a first optical sensor, an electrical stimulator, and a controller. The controller can be configured to: receive a sensing signal from the first optical sensor, the sensing signal indicative of a kinematic of a first eyelid of a user; and cause the electrical stimulator to emit a first electrical stimulus in response to the sensing signal, the first electrical stimulus configured to cause a second eyelid of the user to close.

In any of the embodiments disclosed herein, the system can further comprise a frame. The first optical sensor can be coupled to the frame, such that when the frame is worn by the user, the first optical sensor is positioned proximate the first eyelid of the user.

In any of the embodiments disclosed herein, the electrical stimulator can be coupled to the frame, such that when the frame is worn by a user, the electrical stimulator is configured to provide epicutaneous stimulation of an orbicularis oculi muscle corresponding to the second eyelid of the user.

In any of the embodiments disclosed herein, the frame can be an eyeglass frame.

In any of the embodiments disclosed herein, the first optical sensor can comprise one or more photodiodes and/or one or more image sensors.

In any of the embodiments disclosed herein, the first optical sensor can be configured to sense visible and/or infrared light.

In any of the embodiments disclosed herein, the electrical stimulator can comprise a first electrode and a second electrode, and the controller can be configured to cause the electrical stimulator to apply an electric potential between the first and second electrodes.

In any of the embodiments disclosed herein, at least one of the first electrode and second electrode can be positioned proximate a nasal bridge of the user when the eyelid closure system is worn by the user.

In any of the embodiments disclosed herein, at least one of the first electrode and second electrode can be positioned proximate a temple of the user when the eyelid closure system is worn by the user.

In any of the embodiments disclosed herein, the system can further comprise an eyeglass frame, and the first and second electrodes can be positioned on the eyeglass frame.

In any of the embodiments disclosed herein, the system can further comprise a second optical sensor. The controller can be further configured to receive a second sensing signal from the second optical sensor. The second sensing signal can be indicative of a kinematic of the second eyelid of a user.

In any of the embodiments disclosed herein, the controller can be configured to cause the electrical stimulator to emit a second electrical stimulus in response to the second sensing signal being indicative that no kinematic of the second eyelid is detected. The second electrical stimulus can have a magnitude greater than a magnitude of the first electrical stimulus.

Another embodiment of the present disclosure provides an eyelid closure system, comprising a frame, a first optical sensor, electrical stimulator, and a controller. The frame can be configured to be worn on the head of a user. The first optical sensor can be coupled to the frame and configured to generate a first signal indicative of a kinematic of a first eyelid of the user. The electrical stimulator can be coupled to the frame. The electrical stimulator can comprise first and second electrodes. The controller can be configured to: receive the first signal from the first optical sensor; determine, based on the first signal whether the first eyelid of the user is moving; and if the first eyelid of the user is moving, cause the electrical stimulator to apply a first electric potential between the first and second electrodes.

In any of the embodiments disclosed herein, the system can further comprise a second optical sensor coupled to the frame and configured to generate a second signal indicative of a kinematic of the second eyelid of the user. The controller can be further configured to: receive the second signal from the second optical sensor; determine, based on the second signal whether the second eyelid of the user is closed; and if the second eyelid of the user is not closed, adjust a stimulus strength of the electrical stimulator.

These and other aspects of the present disclosure are described in the Detailed Description below and the accompanying drawings. Other aspects and features of embodiments will become apparent to those of ordinary skill in the art upon reviewing the following description of specific, exemplary embodiments in concert with the drawings. While features of the present disclosure may be discussed relative to certain embodiments and figures, all embodiments of the present disclosure can include one or more of the features discussed herein. Further, while one or more embodiments may be discussed as having certain advantageous features, one or more of such features may also be used with the various embodiments discussed herein. In similar fashion, while exemplary embodiments may be discussed below as device, system, or method embodiments, it is to be understood that such exemplary embodiments can be implemented in various devices, systems, and methods of the present disclosure.

### BRIEF DESCRIPTION OF THE DRAWINGS

The following detailed description of specific embodiments of the disclosure will be better understood when read in conjunction with the appended drawings. For the purpose of illustrating the disclosure, specific embodiments are shown in the drawings. It should be understood, however, that the disclosure is not limited to the precise arrangements and instrumentalities of the embodiments shown in the drawings.

FIG. 1 provides a perspective view of an eyelid closure system, in accordance with some embodiments of the present disclosure.

FIG. 2 provides a block diagram of a computing device for use with some embodiments of the present disclosure.

### DETAILED DESCRIPTION

To facilitate an understanding of the principles and features of the present disclosure, various illustrative embodiments are explained below. The components, steps, and materials described hereinafter as making up various elements of the embodiments disclosed herein are intended to be illustrative and not restrictive. Many suitable components, steps, and materials that would perform the same or similar functions as the components, steps, and materials described herein are intended to be embraced within the scope of the disclosure. Such other components, steps, and materials not described herein can include, but are not limited to, similar components or steps that are developed after development of the embodiments disclosed herein.

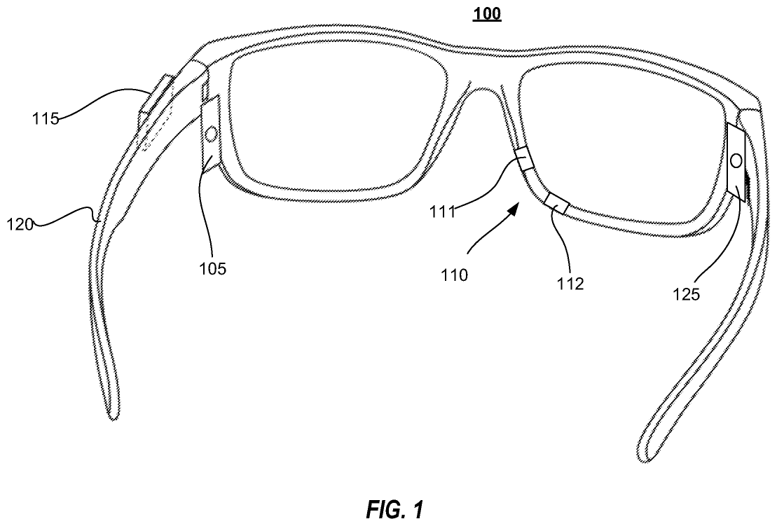

As shown in FIG. 1, an exemplary embodiment of the present disclosure provides an eyelid closure system 100 comprising a first optical sensor 105, an electrical stimulator 110, and a controller 115. In some embodiments, as shown in FIG. 1, the system 100 can further comprise a frame 120 supporting the sensor 105, stimulator 110, and controller 115. The frame 120 can be any mechanical frame structure that can be worn by a user, such that, when worn, the sensor(s) 105 and stimulator 110 are positioned to provide the various functions described below.

The first optical sensor 105 can be many different optical sensors or sensor systems known in the art, including, but not limited to one or more photodiodes, image sensors (e.g., camera), and the like. The optical sensor 105 can detect various wavelengths of light, including, but not limited to, visible light, infrared light, and the like. The first optical sensor 105 can be used to determine when an eyelid of a user begins to close. For example, if a user has one eye that the user cannot close or struggles with closing, i.e., a “bad eye,” and a user with substantially normal operation, i.e., a “good eye,” the first optical sensor 105 can be used to determine when the good eye of the user is closed or begins to close. This can be done by detecting a kinematic of the user's eyelid. For example, the optical sensor 105 can comprise one or more photodiodes that detect light reflected from the area proximate the eye of the user. Thus, when the user's eyelid is in the open position, the amount of light detected by the sensor can be greater than when the user's eyelid is closed or beginning to close. Similarly, if the optical sensor 105 comprises an imaging sensor, the sensor can collect images of the users eye, which can be analyzed to determine if the user's eye lid is open, closed, or beginning to close.

The system 100 can further comprise an electrical stimulator 110. The stimulator 110 can be configured to deliver an electrical stimulus to one or more muscles surrounding the user's eye to cause the user's eye to close. For example, stimulator 110 can be configured to provide epicutaneous (or transcutaneous) stimulation of an orbicularis oculi muscle of the user. The electrical stimulator 110 can be many electrical stimulators known in the art. In some embodiments, the electrical stimulator 110 can comprise one or more electrodes 111 112 (such as patch electrodes). Although the system 100 shown in FIG. 1 includes two electrodes 111 112, various numbers of electrodes are contemplated by the present disclosure. The electrodes 111 112 can be positioned on the frame 120 in appropriate locations to deliver the desired electrical stimulus. For example, in some embodiments, the at least one electrode can be positioned proximate a nasal bridge of the user when the eyelid closure system 100 is worn by the user. In some embodiments, at least one electrode can be positioned proximate a temple of the user when the eyelid closure system 100 is worn by the user.

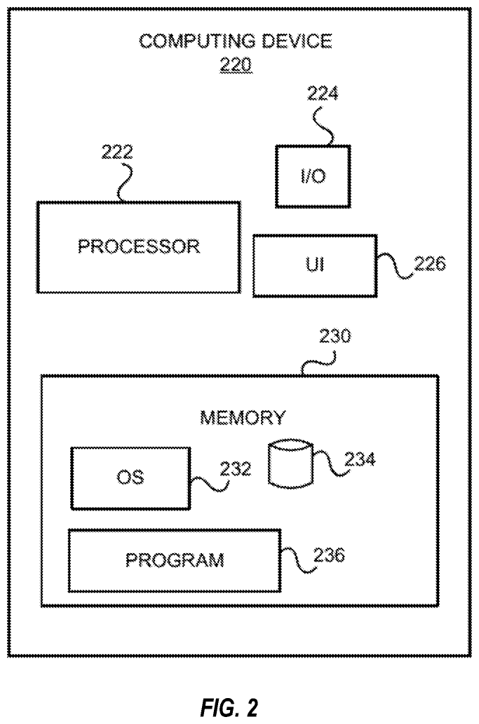

The system 100 can further comprise a controller 115. The controller 115 can be many controllers known in the art. In some embodiments, the controller 115 can comprise one or more components of the computing device shown in FIG. 2 (discussed in detail below). The controller 115 can be positioned about the frame of the system 100. In some embodiments, the controller 115 can be embedded within the frame.

The controller 115 can be configured to receive a sensing signal from the first optical sensor. The controller 115 can process the sensing signal to determine a kinematic of the user's eyelid, for example, is the eyelid open, closed, or closing. If the controller 115 determines the user's eyelid is closed or beginning to close (e.g., detects movement of the eyelid), the controller 115 can cause the electrical stimulator 110 to emit an electrical stimulus configured to cause a second eyelid of the user to close. For example, the controller 115 can be configured to cause the electrical stimulator 110 to apply an electric potential between the first and second electrodes 111 112 to stimulate a muscle causing the eyelid to close. The stimulus provided can be any many different forms. For example, in some embodiments, the stimulus can be provided by a single electrical pulse or a series of pulses. Additionally, the time duration of the stimulus can vary, in accordance with various embodiments of the present disclosure.

The controller 115 can further be configured to communicate wirelessly, e.g., Bluetooth, with a remote device (e.g., remote computer, smartphone, tablet, server, etc.). For example, the controller 115 can send data to the remote device indicative of the operation of the system 100.

As discussed above, as shown in FIG. 1, the system 100 can comprise a frame 120. The frame 120 can be in many different shapes, in accordance with various embodiments of the present disclosure. As shown in FIG. 1, the frame 120 can be an eyeglass frame. In some embodiments, one or more components of the system 100 can be embedded, at least partially, within the frame 120, to create a more aesthetic appearance for the system 100. For example, the sensor 105 can be partially embedded within a portion of the frame 120 proximate the “good eye,” and the one or more electrodes 111 112 can be partially embedded within the frame 120 proximate the “bad eye.” Similarly, the controller 115 can be embedded within a side portion of the frame 120 so that it is not easily viewable by others when the frame 120 is worn by the user.

Though not shown in FIG. 1 the system 100 can comprise various electrical connections to connect the controller 115 to the sensors 105 and/or stimulator 110. In some embodiments, these electrical connections, e.g., wires, can also be embedded, at least partially, within the frame 120.

In some embodiments, the system 100 can further comprise a second optical sensor 125 to monitor the effect of the stimulator on closing the “bad eye.” The second optical sensor 125, like the first optical sensor 105, can be many different optical sensors known in the art. The second optical sensor 120 can monitor a kinematic of the second eye to determine if the second eye is closed or closing. The controller 115 can receive a sensing signal from the second optical sensor 125. If the controller 115 determines, from the sensing signal from the second optical sensor 125, that the stimulus did not result in the second eye closing, the controller 115 can adjust the stimulus provided by the electrical stimulator, e.g., increase a magnitude of the electric potential applied to the electrodes 111 112.

FIG. 2 illustrates an exemplary computing device. The controller 115 can comprise one or more components of the computing device to perform the various functions of the controller discussed above. As shown, the computing device 220 may include a processor 222, an input/output (“I/O”) device 224, a memory 230 containing an operating system (“OS”) 232 and a program 236. In certain example implementations, the computing device 220 may be a microcontroller or a processor to perform one or more of the processes and functionalities associated with the disclosed embodiments. In some embodiments, the computing device 220 may further include a peripheral interface, a transceiver, a mobile network interface in communication with the processor 222, a bus configured to facilitate communication between the various components of the computing device 220, and a power source configured to power one or more components of the computing device 220.

A peripheral interface, for example, may include the hardware, firmware and/or software that enable(s) communication with various peripheral devices, such as media drives (e.g., solid state), other processing devices, or any other input source used in connection with the disclosed technology. In some embodiments, a peripheral interface may include a serial port, a parallel port, a general-purpose input and output (GPIO) port, a game port, a universal serial bus (USB), a micro-USB port, a high definition multimedia interface (HDMI) port, a video port, an audio port, a Bluetooth™ port, a near-field communication (NFC) port, another like communication interface, or any combination thereof.

In some embodiments, a transceiver may be configured to communicate with compatible devices and ID tags when they are within a predetermined range. A transceiver may be compatible with one or more of: radio-frequency identification (RFID), near-field communication (NFC), Bluetooth™, low-energy Bluetooth™ (BLE), WiFi™, ZigBee™, ambient backscatter communications (ABC) protocols or similar technologies.

A mobile network interface may provide access to a cellular network, the Internet, or another wide-area or local area network. In some embodiments, a mobile network interface may include hardware, firmware, and/or software that allow(s) the processor(s) 222 to communicate with other devices via wired or wireless networks, whether local or wide area, private or public, as known in the art. A power source may be configured to provide an appropriate alternating current (AC) or direct current (DC) to power components.

The processor 222 may include one or more of a microprocessor, microcontroller, digital signal processor, co-processor or the like or combinations thereof capable of executing stored instructions and operating upon stored data. The memory 230 may include, in some implementations, one or more suitable types of memory (e.g. such as volatile or non-volatile memory, random access memory (RAM), read only memory (ROM), programmable read-only memory (PROM), erasable programmable read-only memory (EPROM), electrically erasable programmable read-only memory (EEPROM), hard disks, removable cartridges, flash memory, and the like), for storing files including an operating system, application programs (including, for example, a web browser application, a widget or gadget engine, and or other applications, as necessary), executable instructions and data. In one embodiment, the processing techniques described herein may be implemented as a combination of executable instructions and data stored within the memory 230.

The processor 222 may be one or more known processing devices for embedded designs, such as, but not limited to, a microprocessor from the ARM architecture family or a microcontroller. The processor 222 may constitute a single core or multiple core processor that executes parallel processes simultaneously. For example, the processor 222 may be a single core processor that is configured with virtual processing technologies. In certain embodiments, the processor 222 may use logical processors to simultaneously execute and control multiple processes. The processor 222 may implement virtual machine technologies, or other similar known technologies to provide the ability to execute, control, run, manipulate, store, etc. multiple software processes, applications, programs, etc. The processor 222 may also comprise multiple processors, each of which is configured to implement one or more features/steps of the disclosed technology. One of ordinary skill in the art would understand that other types of processor arrangements could be implemented that provide for the capabilities disclosed herein.

In accordance with certain example implementations of the disclosed technology, the computing device 220 may include one or more storage devices configured to store information used by the processor 222 (or other components) to perform certain functions related to the disclosed embodiments. In one example, the computing device 220 may include the memory 230 that includes instructions to enable the processor 222 to execute one or more applications, such as server applications, network communication processes, and any other type of application or software known to be available on computer systems. Alternatively, the instructions, application programs, etc. may be stored in an external storage or available from a memory over a network. The one or more storage devices may be a volatile or non-volatile, semiconductor, removable, non-removable, or other type of storage device or tangible computer-readable medium.

In one embodiment, the computing device 220 may include a memory 230 that includes instructions that, when executed by the processor 222, perform one or more processes consistent with the functionalities disclosed herein. Methods, systems, and articles of manufacture consistent with disclosed embodiments are not limited to separate programs or computers configured to perform dedicated tasks. For example, the computing device 220 may include the memory 230 that may include one or more programs 236 to perform one or more functions of the disclosed embodiments.

The processor 222 may execute one or more programs located remotely from the computing device 220. For example, the computing device 220 may access one or more remote programs that, when executed, perform functions related to disclosed embodiments.

The memory 230 may include one or more memory devices that store data and instructions used to perform one or more features of the disclosed embodiments. The memory 230 may also include any combination of one or more databases controlled by memory controller devices (e.g., server(s), etc.) or software. The memory 230 may include software components that, when executed by the processor 222, perform one or more processes consistent with the disclosed embodiments. In some examples, the memory 230 may include a database 234 configured to store various data described herein. For example, the database 234 can be configured to store the software repository 102 or data generated by the repository intent model 104 such as synopses of the computer instructions stored in the software repository 102, inputs received from a user (e.g., responses to questions or edits made to synopses), or other data that can be used to train the repository intent model 104.

The computing device 220 may also be communicatively connected to one or more memory devices (e.g., databases) locally or through a network. The remote memory devices may be configured to store information and may be accessed and/or managed by the computing device 220. By way of example, the remote memory devices may be document management systems, Microsoft™ SQL database, SharePoint™ databases, Oracle™ databases, Sybase™ databases, or other relational or non-relational databases. Systems and methods consistent with disclosed embodiments, however, are not limited to separate databases or even to the use of a database.

The computing device 220 may also include one or more I/O devices 224 that may comprise one or more user interfaces 226 for receiving signals or input from devices and providing signals or output to one or more devices that allow data to be received and/or transmitted by the computing device 220. For example, the computing device 220 may include interface components, which may provide interfaces to one or more input devices, such as one or more keyboards, mouse devices, touch screens, track pads, trackballs, scroll wheels, digital cameras, microphones, sensors, and the like, that enable the computing device 220 to receive data from a user.

In example embodiments of the disclosed technology, the computing device 220 may include any number of hardware and/or software applications that are executed to facilitate any of the operations. The one or more I/O interfaces may be utilized to receive or collect data and/or user instructions from a wide variety of input devices. Received data may be processed by one or more computer processors as desired in various implementations of the disclosed technology and/or stored in one or more memory devices.

While the computing device 220 has been described as one form for implementing the techniques described herein, other, functionally equivalent, techniques may be employed. For example, some or all of the functionality implemented via executable instructions may also be implemented using firmware and/or hardware devices such as application specific integrated circuits (ASICs), programmable logic arrays, state machines, field programmable gate array (FPGAs) etc. Furthermore, other implementations of the computing device 220 may include a greater or lesser number of components than those illustrated.

It is to be understood that the embodiments and claims disclosed herein are not limited in their application to the details of construction and arrangement of the components set forth in the description and illustrated in the drawings. Rather, the description and the drawings provide examples of the embodiments envisioned. The embodiments and claims disclosed herein are further capable of other embodiments and of being practiced and carried out in various ways. Also, it is to be understood that the phraseology and terminology employed herein are for the purposes of description and should not be regarded as limiting the claims.

Accordingly, those skilled in the art will appreciate that the conception upon which the application and claims are based may be readily utilized as a basis for the design of other structures, methods, and systems for carrying out the several purposes of the embodiments and claims presented in this application. It is important, therefore, that the claims be regarded as including such equivalent constructions.

Furthermore, the purpose of the foregoing Abstract is to enable the United States Patent and Trademark Office and the public generally, and especially including the practitioners in the art who are not familiar with patent and legal terms or phraseology, to determine quickly from a cursory inspection the nature and essence of the technical disclosure of the application. The Abstract is neither intended to define the claims of the application, nor is it intended to be limiting to the scope of the claims in any way.

1. An eyelid closure system comprising:
a first optical sensor;
an electrical stimulator; and
a controller configured to:
receive a sensing signal from the first optical sensor, the sensing signal indicative of a kinematic of a first eyelid of a user; and
cause the electrical stimulator to emit a first electrical stimulus in response to the sensing signal, the first electrical stimulus configured to cause a second eyelid of the user to close.

2. The eyelid closure system of claim 1 further comprising a frame;
wherein the first optical sensor is coupled to the frame, such that when the frame is worn by the user, the first optical sensor is positioned proximate the first eyelid of the user.

3. The eyelid closure system of claim 2, wherein the electrical stimulator is coupled to the frame, such that when the frame is worn by a user, the electrical stimulator is configured to provide epicutaneous stimulation of an orbicularis oculi muscle corresponding to the second eyelid of the user.

4. The eyelid closure system of claim 2, wherein the frame is an eyeglass frame.

5. An eyelid closure system comprising:
a first optical sensor configured to generate a first signal indicative of a kinematic of a first eyelid of the user;
a second optical sensor configured to generate a second signal indicative of a kinematic of the second eyelid of the user;
an electrical stimulator; and
a controller configured to:
receive the first and second signals;
cause the electrical stimulator to emit a first electrical stimulus in response to the first signal, the first electrical stimulus intended to cause the second eyelid of the user to close;
determine, based on the second signal, if the second eyelid of the user is closed in response to the first electrical stimulus; and
if the second eyelid of the user is not closed, adjust a stimulus strength of the electrical stimulator.

6. The eyelid closure system of claim 5, wherein the first optical sensor is configured to sense visible and/or infrared light.

7. The eyelid closure system of claim 5, wherein the electrical stimulator comprises a first electrode and a second electrode; and
wherein the controller is further configured to cause the electrical stimulator to apply an electric potential between the first and second electrodes.

8. The eyelid closure system of claim 7, wherein at least one of the first electrode and the second electrode is positioned proximate a nasal bridge of the user when the eyelid closure system is worn by the user.

9. The eyelid closure system of claim 7, wherein at least one of the first electrode and the second electrode is positioned proximate a temple of the user when the eyelid closure system is worn by the user.

10. The eyelid closure system of claim 5 further comprising an frame;
wherein at least one of:
the first optical sensor comprises one or more photodiodes and/or one or more image sensors;
the first optical sensor is coupled to the frame and positioned proximate the first eyelid of the user;
the second optical sensor is coupled to the frame and positioned proximate the second eyelid of the user;
the first and second optical sensors are coupled to the frame;
the electrical stimulator is coupled to the frame, such that when the frame is worn by the user, the electrical stimulator is configured to provide epicutaneous stimulation of an orbicularis oculi muscle corresponding to the second eyelid of the user; or
the frame is an eyeglass frame.

11. (canceled)

12. The eyelid closure system of claim 5, wherein adjusting the stimulus strength of the electrical stimulator comprises further configuring the controller to cause the electrical stimulator to emit a second electrical stimulus, the second electrical stimulus having a magnitude greater than a magnitude of the first electrical stimulus.

13. An eyelid closure system comprising:
a frame configured to be worn on the head of a user;
a first optical sensor coupled to the frame and configured to generate a first signal indicative of a kinematic of a first eyelid of the user;
a second optical sensor coupled to the frame and configured to generate a second signal indicative of a kinematic of the second eyelid of the user;
an electrical stimulator coupled to the frame, the electrical stimulator comprising first and second electrodes; and
a controller configured to:
receive the first signal from the first optical sensor;
receive the second signal from the second optical sensor;
determine, based on the first signal whether the first eyelid of the user is moving;
determine, based on the second signal whether the second eyelid of the user is closed;
if the first eyelid of the user is moving, cause the electrical stimulator to apply a first electric potential between the first and second electrodes; and
if the second eyelid of the user is not closed, adjust a stimulus strength of the electrical stimulator.

14. The eyelid closure system of claim 13, wherein the frame is an eyeglass frame.

15. The eyelid closure system of claim 13, wherein the first optical sensor comprises one or more photodiodes and/or one or more image sensors.

16. The eyelid closure system of claim 13, wherein the first optical sensor is configured to sense infrared light and/or visible light.

17. The eyelid closure system of claim 13, wherein, when the frame is worn by the user, at least one of the first electrode and second electrode is positioned proximate a nasal bridge of the user.

18. The eyelid closure system of claim 13, wherein, when the frame is worn by the user, at least one of the first electrode and second electrode is positioned proximate a temple of the user.

19. (canceled)
